(19) United States
(12) Patent Application Publication
(10) Pub. No.: US 2008/0305100 A1
(43) Pub. Date: Dec. 11, 2008

(54) Activated Protein C Inhibits Undesirable Effects of Plasminogen Activator in the Brain

(76) Inventors: Berislav V. Zlokovic, Rochester, NY (US); Dong Liu, Rochester, NY (US); Tong Cheng, Webster, NY (US); Huang Guo, Pittsford, NY (US)

Correspondence Address: NIXON & VANDERHYE, PC, 901 NORTH GLEBE ROAD, 11TH FLOOR, ARLINGTON, VA, 22203, US

(21) Appl. No.: 11/632,850

(22) PCT Filed: Jul. 25, 2005
(86) PCT No.: PCT/US05/26202

§ 371 Date: Aug. 30, 2007

Related U.S. Application Data
(60) Provisional application No. 60/590,339, filed on Jul. 23, 2004.

(51) Int. Cl. A61K 38/48; C40B 30/06; A61P 9/00

(52) U.S. Cl. 424/94.64; 506/10

(57) ABSTRACT

Activated protein C (APC), a prodrug, and/or a variant of APC may be used to inhibit undesirable effects of plasminogen activator: e.g., apoptosis or cell death of neurons and endothelial cells, brain hemorrhage or intracerebral bleeding, and/or tissue damage in a subject's brain. Inhibition appears to act through the extrinsic pathway of death receptor signal transduction. This represents an improvement in treatment using plasminogen activator (e.g., fibrinolysis). By reducing undesirable effects, the window for fibrinolytic therapy by plasminogen activator may be widened.

### CROSS REFERENCE TO RELATED APPLICATION

This application claims the benefit of provisional U.S. Patent Appln. No. 60/590,339, filed Jul. 23, 2004.

### STATEMENT REGARDING FEDERALLY-SPONSORED RESEARCH

The U.S. Government has certain rights in this invention as provided by NIH grant HL63290 from the Department of Health and Human Services.

### BACKGROUND OF THE INVENTION

This invention relates to the use of activated protein C (APC), a prodrug, and/or a variant of APC to inhibit undesirable effects of plasminogen activator in the brain.

Most strokes are ischemic and thrombotic in nature. Thrombolytic therapy with tissue-type plasminogen activator (tPA), currently the only FDA-approved treatment for acute ischemic stroke^{1,2}, is problematic because tPA is capable of damaging neurons³⁻⁷ and increasing symptomatic brain hemor-rhage⁸. Despite an urgent need to improve the effectiveness of tPA and inhibit the undesirable effects thereof⁹, the mechanism of tPA mediating such effects in the brain were not fully understood. Whether tPA itself is directly toxic to neurons⁴ or if its toxicity is secondary to plasmin generation⁶ was unknown. The undesirable effects of tPA on the blood-brain barrier (BBB) include opening of tight junctions¹⁰ and activation of matrix metalloproteinases (MMPs)¹¹, which are critical for development of hemorrhage¹²⁻¹⁴. It was also not known whether tPA is directly toxic to cells of ischemic brain endothelium, which forms the BBB In vivo.

Activated protein C (APC), a serine protease with systemic anticoagulant and anti-inflammatory activities, has remarkable direct cellular anti-apoptotic activities^{15,16}. The protein C pathway is linked to ischemic brain injury by clinical and biochemical evidence^{15,16}. APC reduces organ damage in animal models of sepsis¹⁶ and humans with severe sepsis¹⁷, represses apoptosis in the developing placenta 8, and protects brain during transient ischemia^{19,20}. APC alters endothelial gene expression profiles^{21,22} and upregulates the anti-apoptotic Bcl-2 homolog A1 and inhibitor of apoptosis protein-1^{21,22}. APC also blocks p53-dependent apoptosis in ischemic brain endothelial cells (BEC)²⁰, caspase-8 activation in staurosporine-mediated neuronal apoptosis²³, and caspase-3-dependent nuclear translocation of apoptosis inducing factor (AIF) during N-methyl-D-aspartate (NMDA)-mediated apoptosis²³. Thus, APC may be directly neuronal protective in stroke and other neurodegenerative disorders²⁴. Whether APC was protective during thrombolysis, however, was not known.

Here, we demonstrate that tPA induces apoptosis in ischemic human BEC and in an NMDA model of neuronal excitotoxic injury by activating caspase-8, rather than by mitochondria-dependent activation of caspase-9 which normally mediates injury in these brain cells in the absence of tPA^{20,23} APC blocked tPA-induced apoptosis in ischemic BEC and in NMDA-treated neurons by inhibiting the caspase-8 activation upstream of caspase-3 and upstream of AIF nuclear translocation, respectively. APC blocked tPA/hypoxia-mediated activation of MMP-9 in BEC, which may be involved in an early disruption of the BBB preceding neurotoxicity and hemorrhage after tPA treatment¹²⁻¹⁴. In a mouse model of stroke, APC limited tPA-induced cerebral injury and hemorrhage in vivo consistent with its substantial anti-apoptotic effects on tPA-mediated apoptosis in hypoxic neurons and BEC in vitro. Thus, APC diminishes tPA's direct toxicity on brain cells in vitro and in vivo, as well as tPA-mediated disruption of the BBB, suggesting APC is an ideal neuroprotectant candidate for tPA adjunctive therapy for ischemia.

It is an objective of the invention to use activated protein C (APC), prodrugs, and variants thereof in an effective amount to inhibit neurotoxicity and/or hemorrhage in a subject's brain, wherein such undesirable effects are attributable to a plasminogen activator (e.g., tPA).

A long-felt need for improved new therapeutic and prophylactic pharmaceutical compositions (e.g., to reduce or prevent apoptosis and cell death of neurons and the vasculature) is addressed thereby. Also provided are therapeutic and prophylactic methods for inhibition of apoptosis or cell death and promotion of cell survival. Variants of protein C (i.e., a prodrug), variants of activated protein C, formulation strategies, and treatment protocols may be selected for their effect on the caspase-8 signaling pathway or matrix metalloproteinase-9. Processes for using and making the aforementioned products are described. Further objectives and advantages of the invention are described below.

### SUMMARY OF THE INVENTION

The present invention is directed to at least improved treatment of a subject with a plasminogen activator (e.g., fibrinolysis with a tissue-type plasminogen activator) in which at least some of the neuronal and vascular toxicity induced by the plasminogen activator is inhibited by an effective amount of activated protein C (APC). Fibrinolytic treatment has been associated with ischemia and stroke. An effective amount of APC or functional equivalents thereof may be administered to the subject at approximately the same time of such plasminogen activator treatment (or within 24 hours before or after treatment) to provide at least reduced neuronal or vascular toxicity, apoptosis of stressed cells, hemorrhage, tissue damage, or a combination thereof in the brain. For example, APC or a functional equivalent thereof may prevent reduced plasminogen activator-mediated toxicity by acting through the caspase-8 signaling pathway in brain cells (e.g., neurons and endothelial cells) or matrix metalloproteinase-9. Signaling may require an endothelial protein C receptor (EPCR) and/or a protease activated receptor-1 (PAR1) upstream of caspase-8 in the pathway, as well as caspase-3 and/or apoptosis inducing factor downstream of caspase-8 in the pathway. Thus, inhibition appears to act through the extrinsic pathway of death receptor signal transduction.

Therefore, the invention inhibits deleterious effects of a plasminogen activator (e.g., reducing the number or severity of such effects, or preventing their occurrence or worsening) by using activated protein C or a functional equivalent thereof. By reducing deleterious effects, the therapeutic window for treatment with plasminogen activator may be widened (e.g., initiating treatment more than 3 hours after onset of symptoms). Pharmaceutical compositions may be manufactured and assessed in accordance therewith.

Further aspects of the invention will be apparent to persons skilled in the art from the following detailed description and claims, and generalizations thereto.

### BRIEF DESCRIPTION OF THE DRAWINGS

FIG. 1 shows that tPA-induced apoptosis in human BEC and cytoprotection by human APC. FIG. 1A, LDH release from hypoxic and normoxic BEC without (, ▾) or with tPA (◯, ∇). FIG. 1B, Effects of APC () and vehicle (◯) on tPA-induced apoptosis in hypoxic BEC. Caspase-9 (FIG. 1C), caspase-8 (FIG. 1D), or caspase-3 (FIG. 1E) activity in hypoxic and normoxic cells treated with vehicle (, ▾), tPA (◯, ∇) and tPA+APC (▪). FIG. 1G, Effects of caspase-9 or -8 inhibitor on caspase-3 activation in hypoxic BEC+tPA. Mean±s.e.m.; n=3-6.

FIG. 2 shows that tPA potentiates NMDA-mediated apoptosis in mouse cortical neurons through caspase-8. Caspase-9 (FIG. 2A), caspapse-8 (FIG. 2B), or caspase-3 (FIG. 2C) activity in cultured cortical neurons exposed to NMDA or NMDA plus tPA. FIG. 2D, Western blot analysis for p53 in nuclear protein extracts (top) or Bcl-2 and Bax in whole-cell extracts (bottom) in NMDA-treated cells+tPA. FIG. 2E, Quantitation by scanning densitometry of bands in FIG. 2D. Mean±s.e.m.; n=3-5.

FIG. 3 shows that mouse APC or caspase-8 inhibitor blocks tPA-induced NMDA-mediated cortical apoptosis. FIG. 3A, NMDA/tPA-mediated apoptosis in the absence or presence of APC. Caspase-8 (FIG. 3B) or caspase-3 (FIG. 3C) activity in cells treated with tPA plus NMDA in the presence of APC and caspase-8, -9 or -3 inhibitor. FIG. 3D, Western blot analysis of AIF in nuclear extracts from NMDA/tPA treated cells in the presence of APC and caspase-8, -9 or -3 inhibitor. FIG. 3E, Western blot analysis of NR1 and NR2A subunits in membrane fractions 24 h after treatment with tPA. Mean±s.e.m.; n=3-5.

FIG. 4 shows that APC protects against tPA-induced injury during cerebral ischemia in mice. Injury (FIG. 4A), infarction (FIG. 4B), edema volume (FIG. 4C), or motor neurological score (FIG. 4D) in the presence or absence of human tPA and different doses of mouse recombinant APC (mg/kg). tPA and APC were infused intravenously for 30 min during last 10 min of middle cerebral artery occlusion and 20 min into the reperfusion phase. Mean±s.e.m.; n=6 per group.

FIG. 5 shows that tPA and APC affects the neurovasculature during cerebral ischemia in mice. FIG. 5A, Cerebral blood flow (CBF) during middle cerebral artery occlusion and reperfusion in the presence of vehicle (), tPA (▾), or tPA plus mouse recombinant APC (mg/kg) (◯). FIG. 5B, Post-ischemic CBF with tPA or tPA plus APC treatment. Fibrin deposition (FIG. 5C) and CD11b-positive leukocytes (FIG. 5D) in ischemic brain tissue with tPA plus APC. Mean±s.e.m.; n=6 per group.

FIG. 6 shows that tPA-induced hemorrhage and BEC's MMP-9 activity are controlled with APC. The amount of hemoglobin in ischemic hemisphere (FIG. 6A) and hemorrhage (not shown) in the presence or absence of tPA plus APC. In FIG. 6A, mean±s.e.m.; n=6 per group; APC, mouse recombinant APC (mg/kg). FIG. 6B, MMP-9 zymography in normoxic or hypoxic BEC treated with tPA plus APC. FIG. 6C, Dose-dependent effect of APC on pro-MMP-9/MMP-9 activity in hypoxic BEC exposed to tPA. *p<0.01, tPA/hypoxia vs. hypoxia; **p<0.001, APC plus tPA/hypoxia vs. tPA/hypoxia. FIG. 6D, Western blot analysis of ¹²⁵I-tPA in the absence or presence of APC. Mean±s.e.m.; n=3-5/point.

### DESCRIPTION OF SPECIFIC EMBODIMENTS OF THE INVENTION

The present invention is useful in inhibiting deleterious effects of a plasminogen activator (e.g., reducing the number or severity of such effects, or preventing their occurrence or worsening). Known plasminogen activators include alteplase, reteplase, tenecteplase, streptokinase, and urokinase. Inhibition of signaling through caspase-8 by activated protein C (APC) or a variant thereof may be demonstrated by in vitro and in vivo assays (e.g., cell cultures and animal models). Apoptosis and/or cell death may be reduced (or at least mitigated) by the invention. Similarly, hemorrhaging and tissue damage may be reduced or at least mitigated. In particular, neurotoxicity due to overstimulation of N-methyl-D-aspartate (NMDA) receptors is a useful model for neuronal cell injury and death that mimics the effects of neurodegenerative disease. Cytoprotection may be determined at the level of different cell types, organs or tissues, or whole organisms.

The present invention provides methods for inhibiting undesirable effects of a plasminogen activator in a subject's brain. A typical protocol for alteplase is about 0.9 mg/kg body weight of subject (maximum of about 90 mg) administered intravenously over about one minute for about 10% of the dose (i.e., bolus) and then over about one hour for about 90% of the dose (i.e., infusion). It is preferred that treatment of ischemic stroke be initiated within three hours of symptom onset (see Adams et al., Stroke 34:1056-1083, 2003), and more preferably within 90 minutes of symptom onset. But it should be noted that the invention includes initiating treatment with plasminogen activator more than 3 hours after onset of the symptoms of ischemic stroke (e.g., about 6 hours after symptom onset) because undesirable effects of the plasminogen activator are inhibited. For example, an alternative protocol for reteplase is about 0.6 mg/kg body weight of subject administered by an intra-arterial route over about six hours in divided doses of about 8 mg to about 12 mg.

The improvement comprises administering to the subject an effective amount of APC or functional equivalents thereof, thereby inhibiting one or more undesirable effects of plasminogen activator (e.g., apoptotic and other cytotoxic processes in the brain). For example, APC or functional equivalents thereof may be administered at approximately the same time as the initiation of treatment, after treatment with plasminogen activator (e.g., at least 30 minutes, one hour, 90 minutes, 2 hours, 3 hours, 6 hours, 12 hours, or 24 hours after initiation of treatment), or before treatment with plasminogen activator (e.g., at least 30 minutes, one hour, 90 minutes, 2 hours, 3 hours, or 6 hours before initiation of treatment). Neurological damage (e.g., neurons and endothelial cells of the brain) may be at least reduced or limited, and symptoms ameliorated thereby: e.g., brain hemorrhage and tissue damage induced by plasminogen activator, neuronal and vascular toxicities induced by plasminogen activator, apoptosis and cell death of brain cells stressed by ischemic stroke and subsequent treatment with plasminogen activator, and combinations thereof. Efficacy may be evaluated with or without administration of APC or functional equivalents thereof through improved neurological status of subjects and their neurological clinical scores (e.g., NIH scale) after stroke (e.g., Bartlett or Rankin scale); reduced brain hemorrhage (i.e., intracerebral bleeding) or tissue damage induced by plasminogen activator (e.g., swelling and infarction detected using CT or MRI imaging); reduced conversion of ischemic stroke into hemorrhagic stroke which is associated with plasminogen activator treatment (e.g., as detected in the brain by imaging the bleeding using CT or MRI, quantitating hemoglobin in animal models, or simply observing blood); or combinations thereof.

One aspect of the invention includes activated protein C's activities such as an inhibitor of apoptosis or cell death, cell survival factor, and cytoprotective agent. The cell may be derived from brain vessels (e.g., an endothelial cell, a fibroblast, a pericyte, a smooth muscle cell, a veil cell) of a subject, especially from the endothelium of a brain vessel. Alternatively, it may be a neuron, an astrocyte, a microglial cell, or an oligodendrocyte; a precursor or a progenitor cell thereof; or other types of differentiated cell from the subject's central or peripheral nervous system. In particular, “neuron” includes hundreds of different types of neurons, each with distinct properties. Each type of neuron produces and responds to different combinations of neurotransmitters and neurotrophic factors. Neurons typically do not divide in the adult brain, nor do they generally survive long in vitro. The method of the invention provides for the protection from death or senescence of neurons from virtually any region of the brain and spinal cord. Neurons include those in embryonic, fetal, or adult neural tissue, including tissue from the hippocampus, cerebellum, spinal cord, cortex (e.g., motor or somatosensory cortex), striatum, basal forebrain (e.g., cholinergic neurons), ventral mesencephalon (e.g., cells of the substantia nigra), and the locus ceruleus (e.g., neuroadrenaline cells of the central nervous system).

Life threatening local and remote tissue damage occurs during stroke because those tissues are deprived for a time of oxygenation (ischemia) then restored with normal circulation (reperfusion). Cell death and tissue damage can lead to organ failure or decreased organ function. The compositions and methodologies of the present invention are useful in treatment of such injury or prevention thereof.

Protein C was originally identified by its anticoagulant and profibrinolytic activities. Upon activation of the zymogen form, activated protein C (APC) is a serine protease which deactivates Factors Vₐ and VIIIₐ. Human protein C is primarily made in the liver as a single polypeptide of 461 amino acids. This precursor molecule is then post-translationally modified by (i) cleavage of a 42 amino acid signal sequence, (ii) proteolytic removal from the one-chain zymogen of the lysine residue at position 155 and the arginine residue at position 156 to produce the two-chain form (i.e., light chain of 155 amino acid residues attached by disulfide linkage to the serine protease-containing heavy chain of 262 amino acid residues), (iii) carboxylation of the glutamic acid residues clustered in the first 42 amino acids of the light chain resulting in nine gamma-carboxyglutamic acid (Gla) residues, and (iv) glycosylation at four sites (one in the light chain and three in the heavy chain). The heavy chain contains the serine protease triad of Asp257, His211 and Ser360.

Similar to most other zymogens of extracellular proteases and the coagulation factors, protein C has a core structure of the chymotrypsin family, having insertions and an N-terminus extension that enable regulation of the zymogen and the enzyme. Of interest are two domains with amino acid sequences similar to epidermal growth factor (EGF). At least a portion of the nucleotide and amino acid sequences for protein C from human, monkey, mouse, rat; hamster, rabbit, dog, cat, goat, pig, horse, and cow are known, as well as mutations and polymorphisms of human protein C (see GenBank accession P04070). Variants of human protein C are known which affect different biological activities.

“Protein C” refers to native genes and proteins belonging to this family as well as variants thereof (e.g., mutations and polymorphisms found in nature or artificially designed). The chemical structure of the genes and proteins may be a polymer of natural or non-natural nucleotides connected by natural or non-natural covalent linkages (i.e., polynucleotide) or a polymer of natural or non-natural amino acids connected by natural or non-natural covalent linkages (i.e., polypeptide). See Tables 1-4 of WIPO Standard ST.25 (1998) for a nonlimiting list of natural and non-natural nucleotides and amino acids. Protein C genes and proteins may be recognized as belonging to this family by comparison to the human homolog PROC, use of nucleic acid binding (e.g., stringent hybridization under conditions of 400 mM NaCl, 40 mM PIPES pH 6.4, 1 mM EDTA, at 50° C. or 70° C. for an oligonucleotide; 500 mM NaHPO₄ pH 7.2, 7% SDS, 1% BSA, 1 mM EDTA, at 45° C. or 65° C. for a polynucleotide of 50 bases or longer; and appropriate washing) or protein binding (e.g., specific immunoassay under stringent binding conditions of 50 mM Tris-HCl pH 7.4, 500 mM NaCl, 0.05% TWEEN 20 surfactant, 1% BSA, at room temperature and appropriate washing); or computer algorithms (Doolittle, Of URFS and ORFS, 1986; Gribskov & Devereux, Sequence Analysis Primer, 1991; and references cited therein).

A “mutation” refers to one or more changes in the sequence of polynucleotides and polypeptides as compared to native protein C, and has at least one function that is more active or less active, an existing function that is changed or absent, a novel function that is not naturally present, or combinations thereof. In contrast, a “polymorphism” also refers to a difference in its sequence as compared to native protein C, but the changes do not necessarily have functional consequences. Mutations and polymorphisms can be made by genetic engineering or chemical synthesis, but the latter is preferred for non-natural nucleotides, amino acids, or linkages. Fusions of domains linked in their reading frames are another way of generating diversity in sequence or mixing-and-matching functional domains. For example, homologous protein C and protein S work best together (e.g., human) and this indicates that their sequences may have coevolved to optimize interactions between the enzyme and its cofactor. Exon shuffling or gene shuffling techniques may be used to select desirable phenotypes in a chosen background (e.g., separable domains with different biological activities, hybrid human/mouse sequences which locate the species determinants).

Percentage identity between a pair of sequences may be calculated by the algorithm implemented in the BESTFIT computer program (Smith & Waterman. J. Mol. Biol. 147:195-197, 1981; Pearson, Genomics 11:635-650, 1991). Another algorithm that calculates sequence divergence has been adapted for rapid database searching and implemented in the BLAST computer program (Altschul et al., Nucl. Acids Res. 25:3389-3402, 1997). In comparison to human sequences, the protein C polynucleotide or polypeptide may be only about 60% identical at the amino acid level, 70% or more identical, 80% or more identical, 90% or more identical, 95% or more identical, 97% or more identical, or greater than 99% identical.

Conservative amino acid substitutions (e.g., Glu/Asp, Val/Ile, Ser/Thr, Arg/Lys, Gln/Asn) may also be considered when making comparisons because the chemical similarity of these pairs of amino acid residues are expected to result in functional equivalency in many cases. Amino acid substitutions that are expected to conserve the biological function of the polypeptide would conserve chemical attributes of the substituted amino acid residues such as hydrophobicity, hydrophilicity, side-chain charge, or size. In comparison to the human sequence, the protein C polypeptide may be only about 80% or more similar, 90% or more similar, 95% or more similar, 97% or more similar, 99% or more similar, or about 100% similar. Functional equivalency or conservation of biological function may be evaluated by methods for structural determination and bioassay. The codons used may also be adapted for translation in a heterologous host by adopting the codon preferences of the host. This would accommodate the translational machinery of the heterologous host without a substantial change in chemical structure of the polypeptide.

Protein C and variants thereof (i.e., deletion, domain shuffling or duplication, insertion, substitution, or combinations thereof) may be used to determine structure-function relationships (e.g., alanine scanning, conservative or nonconservative amino acid substitution). For example, protein C folding and processing, secretion, receptor binding, signaling through EPCR and/or PAR-1, inhibition of caspase-8 signaling, any of the other biological activities described herein, or combinations thereof may be related to changes in the amino acid sequence. See Wells (Bio/Technology 13:647-651, 1995) and U.S. Pat. No. 5,534,617. Directed evolution by directed or random mutagenesis or gene shuffling using protein C may be used to acquire new and improved functions in accordance with selection criteria. Mutant and polymorphic variant polypeptides are encoded by suitable mutant and polymorphic variant polynucleotides. Structure-activity relationships of protein C may be studied (i.e., SAR studies) using variant polypeptides produced with an expression construct transfected in a host cell with or without expressing endogenous protein C. Thus, mutations in discrete domains of protein C may be associated with decreasing or even increasing activity in the protein's function.

Gale et al. (J. Biol. Chem. 277:28836-28840, 2002) have demonstrated that mutations in the surface loops of APC affect its anticoagulant activity. APC mutants KKK191/193AAA (loop 37), RR229/230AA (calcium loop), RR306/312AA (autolysis loop), RKRR306/314AAAA (autolysis loop) have less than about 10%, 5%, 17% and 2% of the anticoagulant activity of native APC, respectively. A more recent study has shown that these APC mutants with reduced anticoagulant activity (i.e., KKK191/193AAA, RR229/230AA, RR306/312AA and RKRR306/312AAAA) retain the anti-apoptotic activity of APC in staurosporine model of apoptosis.

Protein C zymogen, the precursor of activated protein C, is readily converted to activated protein C within the body by proteases. Protein C may be considered a prodrug form of activated protein C. Thus, the use of activated protein C is expressly intended to include protein C and variants thereof. Treatments with protein C would probably require appropriately larger doses known to those of skill in the art (see below).

Recombinant forms of protein C can be produced with a selected chemical structure (e.g., native, mutant, or polymorphic). As an illustration, a gene encoding human protein C is described in U.S. Pat. No. 4,775,624 and can be used to produce recombinant human protein C as described in U.S. Pat. No. 4,981,952. Human protein C can be recombinantly produced in tissue culture and activated as described in U.S. Pat. No. 6,037,322. Natural human protein C can be purified from plasma, activated, and assayed as described in U.S. Pat. No. 5,084,274. The nucleotide and amino acid sequences disclosed in these patents may be used as a reference for protein C.

Doses, dosing protocols, and protein C variants that reduce bleeding in a subject as compared to activated protein C which is endogenous to the subject are preferred. Mutations in the amino acid sequence of native protein C may separate the ability to inhibit (i.e., reduce or prevent) caspase-8 signaling from its other biological activities (e.g., anticoagulant activity). The inhibitory activities of activated protein C may thereby be maintained or increased while decreasing undesirable effects of its administration (e.g., bleeding in the brain and other organs).

Formulations and their Administration

Activated protein C, a prodrug, or a variant thereof may be used to formulate pharmaceutical compositions with one or more of the utilities disclosed herein. They may be administered in vitro to cells in culture, in vivo to cells in the body, or ex vivo to cells outside of a subject which may then be returned to the body of the same subject or another. The cells may be removed from, transplanted into, or be present in the subject (e.g., genetic modification of endothelial cells in vitro and then returning those cells to brain endothelium). Candidate agents may also be screened in vitro or in vivo to select those with desirable properties. The cell may be from the endothelium (e.g., endothelial cell, fibroblast, pericyte, smooth muscle cell, veil cell), especially from the endothelium of a brain vessel. It may also be a neuron; a glial cell; a precursor, progenitor, or stem cell thereof; or another differentiated cell from the central or peripheral nervous system.

Use of compositions which comprise a pharmaceutically acceptable carrier (i.e., a vehicle or particulate carrier which is tolerated by the subject and does not cause an unacceptable level of nausea, dizziness, gastric upset, and the like upon its own administration) together with active ingredient dissolved or dispersed therein. It is preferred that the composition not be immunogenic when administered to the subject. Devices and compositions which further comprise components useful for delivering the composition to the subject's brain are known in the art. Addition of such carriers and other components to the composition of the invention is well within the level of skill in this art. For example, a permeable material may release its contents to the local area or a tube may direct the contents of a reservoir to a distant location of the brain.

A pharmaceutical composition may be administered as a formulation which is adapted for direct application to the central nervous system, or suitable for passage through the gut or blood circulation. Alternatively, pharmaceutical compositions may be added to the culture medium. In addition to active ingredient, such compositions may contain pharmaceutically-acceptable carriers and other components known to facilitate administration and/or enhance uptake. It may be administered in a single dose or in multiple doses which are administered at different times. A unit dose of the composition is an amount of the active ingredient which provides neuroprotection, cytoprotection, inhibits apoptosis or cell death, and/or promotes cell survival.

Pharmaceutical compositions may be administered by any known route. By way of example, the composition may be administered by a mucosal, pulmonary, topical, or other localized or systemic route (e.g., enteral or parenteral). In particular, achieving an effective amount of activated protein C, prodrug, or functional variant in the central nervous system may be desired. This may involve a depot injection into or surgical implant within the brain. “Parenteral” includes subcutaneous, intra-arterial, intradermal, intraepidural, intramuscular, intravenous, intrathecal, and other injection or infusion techniques, without limitation. Preferred are bolus and infusion injections by intra-arterial (e.g., carotid artery, middle cerebral artery) or intravenous routes.

Suitable choices in amounts and timing of doses, formulation, and routes of administration can be made with the goals of achieving a favorable response in the subject (i.e., efficacy), and avoiding undue toxicity or other harm thereto (i.e., safety). Thus, “effective” refers to such choices that involve routine manipulation of conditions to achieve a desired effect (e.g., inhibition of neurotoxicity and/or brain hemorrhage). In this manner, “effective amount” refers to the total amount of activated protein C, prodrug (e.g., protein C), or functional variant which achieves the desired effect. Activity can be determined by reference to the amount of APC administered to the subject (e.g., 0.005 mg/kg or less, 0.01 mg/kg or less, 0.05 mg/kg or less, 0.1 mg/kg or less, 0.5 mg/kg or less, 1 mg/kg or less, 2 mg/kg or less); similarly, an “equivalent amount” of prodrug or functional variant can be determined by achieving the same or similar desired effect as the reference amount of activated protein C.

A bolus of the formulation administered only once to a subject is a convenient dosing schedule although achieving an effective concentration of the active ingredient in the brain may require more frequent administration (e.g., three divided injections within 3 hours of symptom onset totaling between 0.1 mg/kg and 1 mg/kg). Acute treatment may involve continuous infusion (e.g., within 3 hours of symptom onset) or a slower infusion (e.g., within 24 hours of symptom onset). The amount of active ingredients in a pharmaceutical composition can also be varied so as to achieve a transient or sustained concentration of the active ingredient or its metabolite in a subject and to result in the desired physiological response. But it is also within the skill of the art to start doses at levels lower than required to achieve the desired physiological effect and to gradually increase the dose until the desired effect is achieved.

The amount of active ingredient administered is dependent upon factors such as, for example, bioactivity and bioavailability of the compound (e.g., half-life in the body, stability, and metabolism); chemical properties of the compound (e.g., molecular weight, hydrophobicity, and solubility); route and scheduling of administration; and the like. It will also be understood that the specific dose level to be achieved for any particular subject may depend on a variety of factors, including age, health, medical history, weight, combination with one or more other drugs, and severity of disease. A typical baseline level of APC in human blood is about 2.2 ng/ml. An effective amount may be sufficient to increase the activity of APC, prodrug, or functional variant equivalent to a rise in blood level of greater than about 1 ng/ml, about 5 ng/ml, or about 50 ng/ml; less than about 0.2 mg/ml, about 0.5 mg/ml, or about 1 mg/ml; or intermediate ranges thereof (e.g., between about 1 ng/ml and about 1 mg/ml) of activated protein C.

In homologous systems (e.g., human native or recombinant APC administered to patients), one or more bolus injections of APC (e.g., 0.005 mg/kg or less, 0.01 mg/kg or less, 0.05 mg/kg or less, 0.1 mg/kg or less, 0.5 mg/kg or less, 1 mg/kg or less, 2 mg/kg or less administered over 1 min) may be sufficient to inhibit the undesirable effects of plasminogen activator without having a significant antithrombotic effect in brain circulation. Infusion of APC at a dose of less than 0.005 mg/kg, less than 0.01 mg/kg, less than 0.05 mg/kg, less than 0.1 mg/kg, less than 0.5 mg/kg, less than 1 mg/kg, or less than 2 mg/kg may also be used. An illustrative amount may be calculated for a 70 kg adult human, and this may be sufficient to treat humans of between 50 kg and 90 kg.

The effective or equivalent amount may be packaged in a “unit dose” with written instructions for achieving one or more desired effects and/or avoiding one or more undesired effects. The aforementioned formulations, routes of administration, and dosing schedules are merely illustrative of the techniques which may be used.

The term “treatment” refers to, inter alia, reducing or alleviating one or more undesirable effects of treatment with plasminogen activator. For example, standard therapy such as stroke treatment with a tissue-type plasminogen activator may be compared with and without APC, prodrug, or a functional variant thereof. This includes therapy of an affected subject or prophylaxis of a subject at risk. For a subject in need of treatment, improvement in a symptom, its worsening, regression, or progression may be determined by objective or subjective measures. The subject in need of treatment may be at risk for or already affected by ischemia or thrombosis; treatment may be initiated before and/or after diagnosis of stroke. In a patient, an indication that treatment is effective may be improved neurological outcome, motor or sensory functions, cognitive functions, psychomotor functions, motor neurological functions, higher integrative intellectual functions, memory, vision, hearing, etc.; reduced brain damage and injury as evidenced by noninvasive image analysis (e.g., MRI or brain perfusion imaging); or combinations thereof. This effect may be confirmed by neuropathological analysis of brain tissue. Ultimately, stabilizing brain endothelial cell functions and preventing their death will lead to improvements in the cerebral blood flow (CBF) and normalization of CBF regulatory functions. In a preclinical study, neurological or behavioral findings, reduction in apoptosis or a marker thereof (e.g., fragmentation or decreased amount of DNA), increased cell survival, decreased cell death, or combinations thereof can be demonstrated in an animal model. These benefits may be achieved with little or no significant system anticoagulation in human or animal subjects. At the cellular level, reduced caspase-8 signaling may be observed. An increase or decrease may be determined by comparison of administration of activated protein C, prodrug, or functional equivalent to placebo (including undesirable effects of fibrinolytic treatment with plasminogen activator).

The present invention may also involve other existing modes of treatment and agents (e.g., protein S, fibrinolytic or antithrombotic agents, steroidal or nonsteroidal anti-inflammatory agents). Thus, combination treatment may be practiced.

### EXAMPLES

While the intravascular thrombolytic effects of tissue-type plasminogen activator (tPA) are beneficial for treatment of ischemic stroke, its neurotoxicity and hemorrhagic potential are problematic. Here, we report that tPA potentiates stress-induced apoptosis in ischemic human brain endothelium and in mouse cortical neurons exposed to N-methyl-D-aspartate via caspase-8 activation which normally plays a minor role or is not involved in apoptosis of these brain cells in the absence of tPA, respectively. APC blocks tPA-induced neurotoxicity in vitro and in vivo, and reduces tPA-mediated cerebral injury and brain hemorrhage in a mouse stroke model. Our results indicate that APC blocks tPA-induced activation of matrix metalloproteinase-9 in brain endothelium and helps maintain the integrity of the blood-brain barrier. These in vitro and in vivo results suggest that APC may add substantially to the effectiveness of tPA treatment of ischemic stroke by counteracting tPA's direct toxic effects on cells in the brain.

### Reagents

Human recombinant tPA (alteplase, Genentech, San Francisco, Calif.) was used. In some in vitro studies we also used human recombinant tPA from Sigma (St. Louis, Mo.). Human plasma-derived APC and mouse recombinant APC were prepared as described^{19,32}. NMDA was from Sigma. For immunoblotting we used polyclonal antibodies against human AIF (1:1000, 1 mg/ml; Chemicon, Temecula, Calif.), human p53 (1:1000, 0.5 mg/ml; Cell Signaling, Beverly, Mass.), human NMDAξ1 (NR1, 1:1000, 0.2 mg/ml; Santa Cruz Biotechnology, Santa Cruz, Calif.), human □-actin (1:2500, 0.2 mg/ml; Santa Cruz Biotechnology), or monoclonal antibody against human Bcl-2 (1:100, 0.2 mg/ml; Santa Cruz Biotechnology) which all crossreact with the corresponding mouse antigens. Polyclonal antibodies against mouse NR2A (1:500, 1 mg/ml; Upstate Biotechnology, Lake Placid, N.Y.) or mouse Bax (1:100, 0.2 mg/ml; Chemicon) was also used. The inhibitory peptides Ac-DEVD-CHO (caspase-3, Sigma), z-IETD-fmk (caspase-8, Sigma), and z-LEHD-fmk (caspase-9, Calbiochem; San Diego, Calif.), were selected on the basis of their substrate specificity^{23,25,26}.

tpA/Hypoxia in Human BEC

Primary human BEC were isolated from rapid autopsies from neurologically normal young individuals after trauma and cultured as we have previously described²⁰. Cells were maintained in serum-free Dulbeco's Modified Eagle Medium and exposed for 1 hr to 16 hr to tPA (20 μg/ml) under normoxic conditions (20% oxygen, 5 mM glucose), hypoxia (<2% oxygen, no glucose), or to tPA plus hypoxia. Hypoxia was induced using an anaerobic chamber (Form a Scientific, Holbrook, N.Y.)²⁰. The levels of O₂ were monitored by O₂ Fyrite (Form a Scientific). z-IETD-fmk (10 μM) or z-LEHD-fmk (15 μM) was applied 2 hr prior to tPA/hypoxia treatment. Human APC (10 nM to 600 nM) was added at the time of tPA/hypoxia treatment. Caspase-3, -8 or -9 activity was determined in the presence of 400 nM APC.

tPA/NMDA-Induced Apoptosis in Neurons

Primary neuronal cultures were prepared from fetal C57BU6J mice as we have previously described²³. Cultures were maintained in serum-free NEUROBASAL medium plus B27 supplement (Gibco BRL, Rockville, Md.) in a humidified 5% CO₂ incubator at 37° C. for 14 days before 3 hr to 24 hr treatment with tPA (20 μg/ml) alone, NMDA (25 μM)/5 μM glycine, or tPA plus NMDA. Ac-DEVD-CHO (50 μM), z-IETD-fmk (20 μM), or z-LEHD-fmk (5 μM) was applied 3 hr prior to tPA/NMDA treatment. Mouse recombinant APC (20 nM) was added 30 min before tPA/NMDA treatment.

### Detection of Cell Injury and Apoptosis

Cell injury in BEC was detected by the release of LDH (LDH assay, Sigma). The Hoechst dye (Hoechst 33,342, Sigma) and in situ terminal deoxynucleotidyl transferase-mediated digoxigenin-dUTP nick-end labeling (TUNEL) (Phoenix Flow Systems, San Diego, Calif.) assays were performed on acetone-fixed BEC or paraformaldexyde-fixed neurons. Images were visualized using a Nikon TE2000-U microscope.

### Caspase-3, -8 and -9 Activities

BEC or cortical neuron lysate was incubated at 37° C. with caspase-3 (DEVD-pNA), caspase-8 (IETD-pNA) (ApoAlert caspase assay kit; Clontech, Palo Alto, Calif.), or caspase-9 (Ac-LEHD-pNA; Chemicon; Temecula, Calif.) substrate. Substrate hydrolysis was determined as absorbance change at 405 nm in a microplate reader²³. Enzymatic activity was expressed in arbitrary units per mg of protein.

### Western Blot Analyses

Whole cellular extracts, nuclear proteins, or cell membrane fractions were prepared as we have previously described²³. The relative abundance of each protein was determined by scanning densitometry using β-actin as an internal control. Data from multiple Western blots (n=3-5) were averaged for statistical analysis.

### MMP Gelatin Zymography

The conditioned medium from BEC exposed to tPA (20 μg/ml) or hypoxia for 4 hr, in the absence or presence of human APC (50 nM to 1,000 nM), was analyzed by gelatin zymography as previously described¹¹. MMP-2 activity did not significantly change. MMP-9 activity was quantified as fold increase compared with normoxic controls.

### In Vivo Stroke Model

Protocols were approved by the Animal Care Committee at the University of Rochester using National Institutes of Health guidelines. Vehicle, tPA (10 mg/kg, 10% bolus/90% infusion), or tPA plus mouse recombinant APC (0.02 mg/kg to 1 mg/kg, 50% bolus/50% infusion) were administered via the femoral vein using separate intravenous lines and our previously described mouse stroke model^{19,20} modified to 45 min MCA occlusion/24 hr reperfusion. tPA was infused during the last 10 min of MCA occlusion and for 20 min into reperfusion. APC was infused either simultaneously with tPA, after tPA infusion, or before tPA infusion. The dose of tPA used here has been frequently used in rodents and is considered to be equivalent to the therapeutic dose in humans^{12,13,44,50}. CBF was monitored by laser Doppler flowmetry (Transonic Systems)¹⁹. Arterial blood gasses were measured¹⁹. Neurological examinations were performed at 24 hr and scored: no neurological deficit (O), failure to extend left forepaw fully (1), turning to left (2), circling to left (3), unable to walk spontaneously (4), or stroke-related death (5). Neuropathological analysis was performed at 24 hr. Unfixed 1-mm coronal brain slices at the level of optic chiasm were incubated in 2% triphenyltetrazolium chloride in phosphate buffer (pH 7.4). Brain injury, infarct, and edema volumes were determined as previously described^{19,20}. Fibrin deposition was quantified by Western blotting with anti-fibrin II antibody (1:500, NYB-T2G1; Accurate Chemical Scientific Corp.)^{19,20,30} and leukocytes were stained with CD11b antibody (1:250, DAKO Corp.)^{19,20}. Hemoglobin was determined by a spectrophotometric assay using Drabkin's reagent (Sigma) as we have previously described¹⁹.

### tPA Iodination

tPA (50 μg) was radioiodinated using IodoBeads (Pierce Chemical, Rockford, Ill.) and 0.5 mCi Na ¹²⁵I (Amersham Biosciences, UK). Free ¹²⁵I was removed by ultrafiltration.

### Statistical Analysis

Data were presented as mean±s.e.m. ANOVA was used to determine statistically significant differences. Non-parametric data (neurological outcome scores) were compared by the Kruskal-Wallis test. p<0.05 was considered statistically significant.

### Example 1

### tPA-Mediated Apoptosis in Ischemic Human BEC and Protection by APC

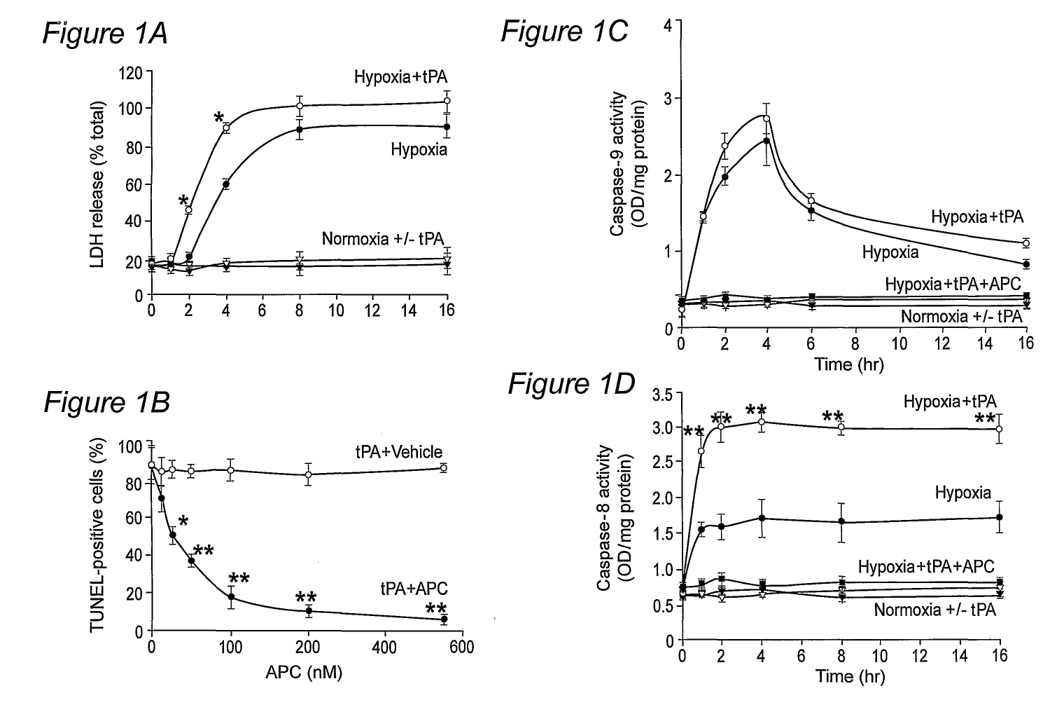

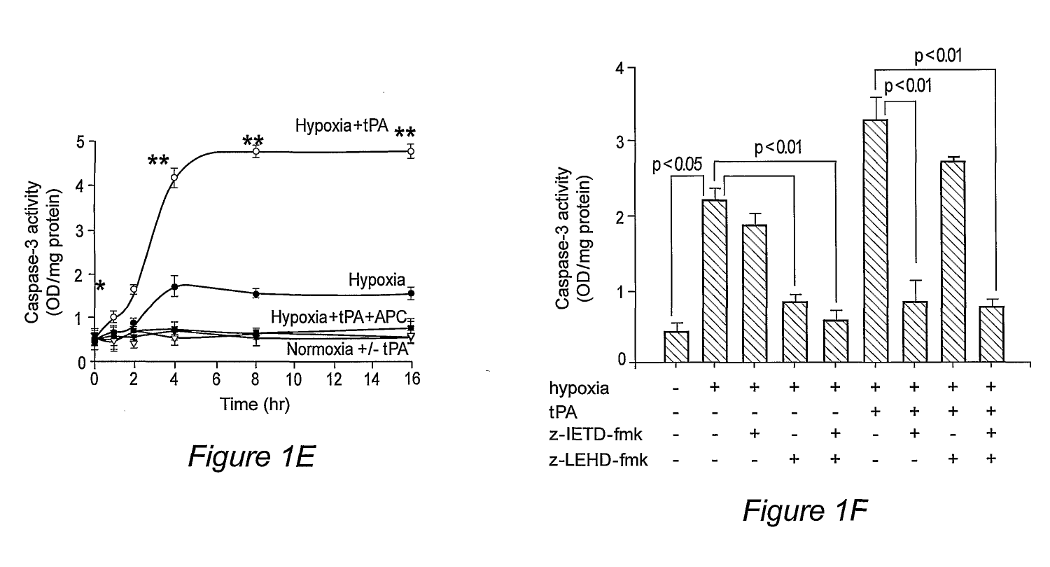

Simultaneous exposure to tPA and hypoxia/aglycemia substantially accelerated BEC injury compared to hypoxia/aglycemia alone. At 2 hr and 4 hr of exposure to tPA, the lactate dehydrogenase (LDH) release into the medium from ischemic BEC increased to 50% and 95% from the background values and 57% in the absence of tPA, respectively (FIG. 1A). tPA alone, however, did not affect LDH release from normoxic BEC. At 4 hr, tPA increased the number of TUNEL-positive hypoxic BEC to 100% from the 60% seen for hypoxia alone. The apoptosis-enhancing effects of tPA were completely blocked by human plasma-derived APC. APC dose-dependently reduced the LDH release and the number of TUNEL-positive tPA-treated hypoxic BEC to almost background levels (FIG. 1B).

Exposure of hypoxic BEC to tPA did not produce a significant increase in caspase-9 activity relative to hypoxia alone (FIG. 1C). In contrast, tPA induced an early and sharp increase in the activation of caspase-8 in ischemic BEC at 1 hr, and remained elevated compared to hypoxia alone over 16 hr (FIG. 1D). These data suggest tPA has a more profound effect on mitochondria-independent compared to mitochondria-mediated apoptotic pathway²⁵. In the presence of tPA there was a significant 2.8-fold increase at 4 hr (p<0.001) and >3.0-fold increase within 16 hr (p<0.001) in caspase-3 activity compared to hypoxia alone (FIG. 1E). APC completely abolished tPA-induced increases in caspase-8 (FIG. 1D) and caspase-3 activity (FIG. 1E) in hypoxic BEC. APC also inhibited caspase-9 activation due to hypoxia (FIG. 1C) consistent with our previous report that APC blocks mitochondria-dependent apoptosis in ischemic BEC²⁰.

To identify caspases required for tPA-induced apoptosis in ischemic BEC, we also studied the effects of caspase-8 and caspase-9 specific inhibitors on caspase-3 activation. In the absence of tPA, the caspase-9 inhibitor z-LEHD-fmk produced greater than 85% inhibition in caspase-3 activation in hypoxic BEC, while caspase-8 inhibitor z-IETD-fmk produced only a modest (less than 15%) inhibition in caspase-3 activation (FIG. 1F). This suggests that the prevailing apoptotic pathway in hypoxia is driven by caspase-9 activation, which is consistent with reported mitochondria-dependent apoptosis of hypoxic BEC²⁰. In contrast, z-LEDH-fmk only slightly inhibited caspase-3 activation in the presence of tPA, whereas z-IEDT-fmk completely (greater than 95%) inhibited caspase-3 activation (FIG. 1F). These results suggest tPA surprisingly shifts the apparent driving force for apoptosis in ischemic BEC from the intrinsic caspase-9-dependent pathway to one or more mechanisms that require activation of caspase-8, the upstream mediator of the death receptor (extrinsic) pathway.

### Example 2

### tpA-Induced Neuronal Apoptosis in NMDA Model and Protection by APC

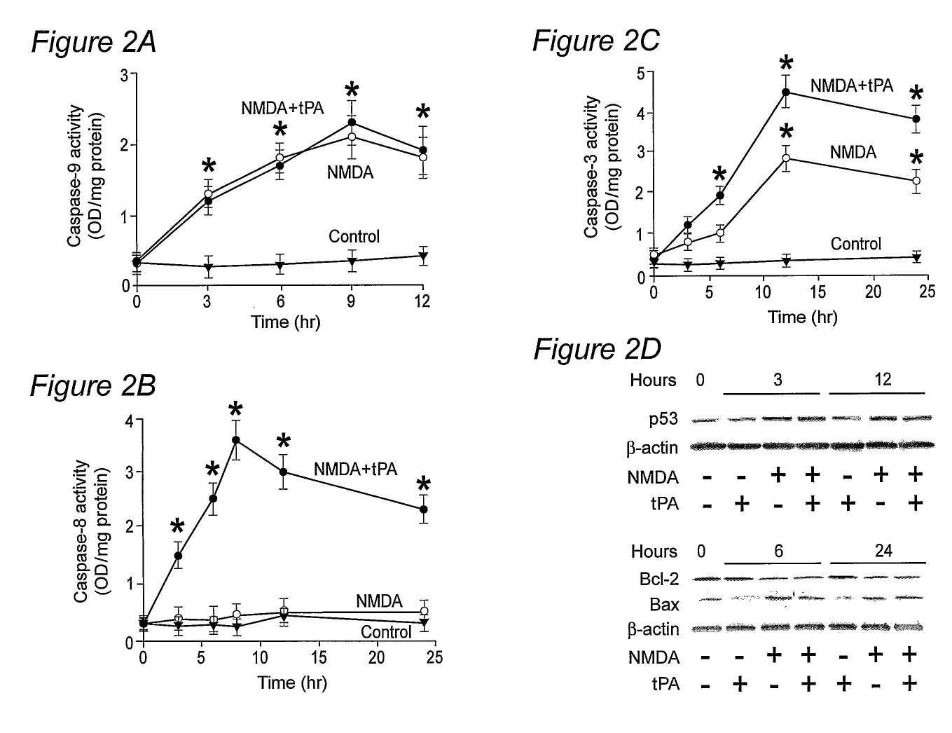

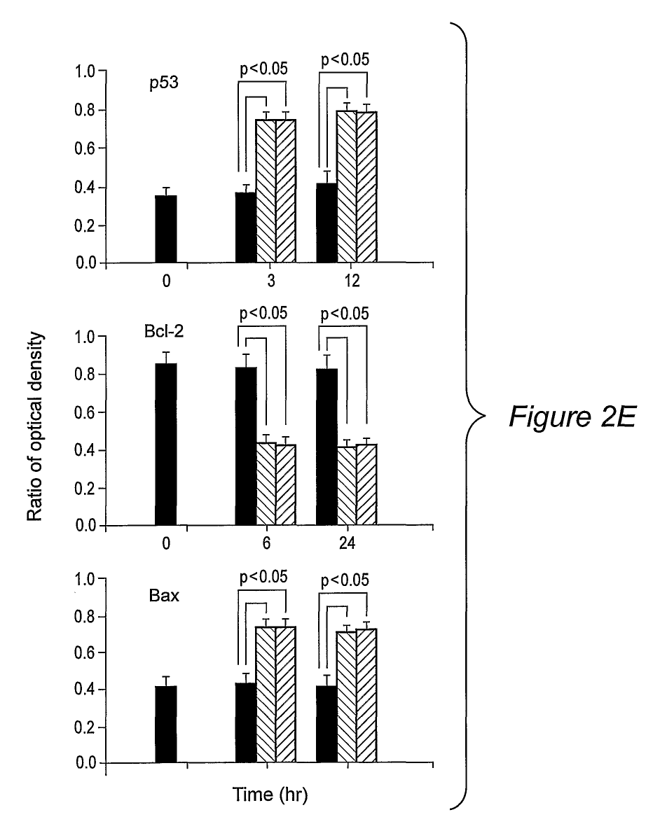

tpA doubles the number of apoptotic neurons 24 hr after exposure to NMDA, which is consistent with a previous report⁴. Compared to NMDA alone, combined tPA/NMDA treatment did not alter caspase-9 activation (FIG. 2A) but resulted in the robust activation of caspase-8 in neurons (FIG. 2B), which is not typically involved in NMDA-mediated apoptosis^{23,26}. tPA increased caspase-3 activation relative to NMDA alone (FIG. 2C). Caspase-3 activation in the NMDA model is typically p53-dependent and mitochondria-mediated through an increased Bax/Bcl-2 ratio^{23,27-29}. We also found that activation of caspase-9 in NMDA-treated neurons (FIG. 2A), but tPA did not affect NMDA-mediated increases in p53 and Bax expression (FIGS. 2D-2E), a decrease in Bcl-2 expression (FIGS. 2D-2E), or caspase-9 activation (FIG. 2A). Thus, tPA did not amplify the known NMDA-mediated mitochondria-dependent proapoptotic effects. tPA alone did not affect neuronal cells or the activation of caspase-8, -9 or -3.

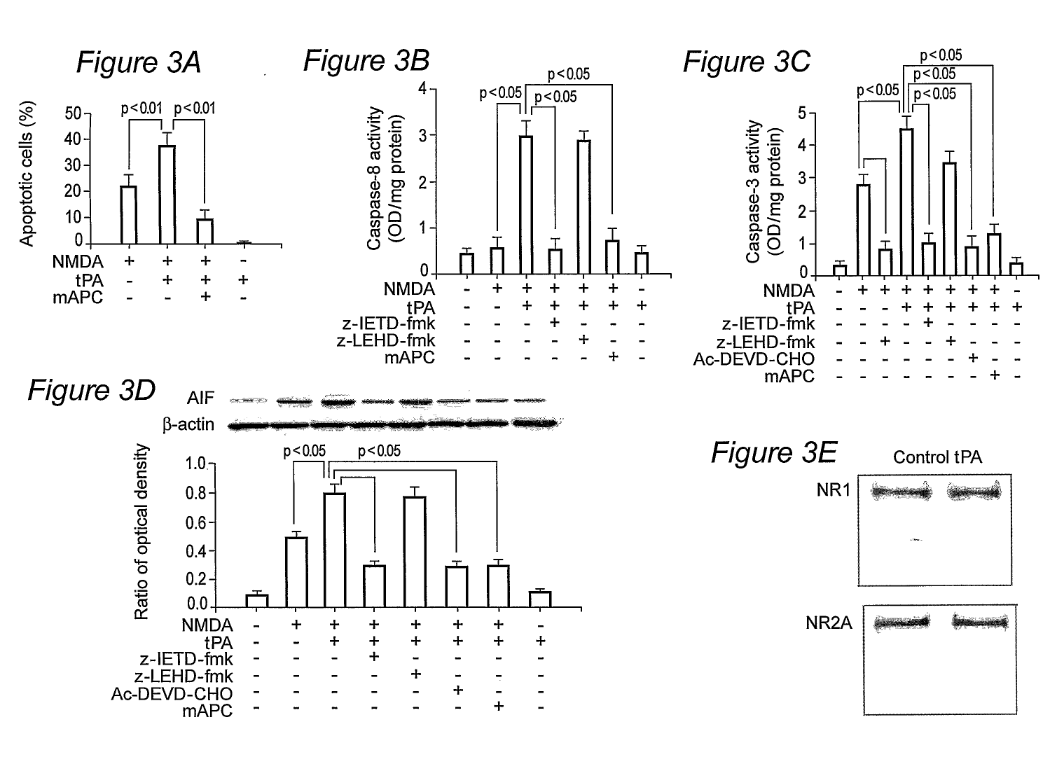

FIG. 3A shows that mouse recombinant APC blocked tPA-induced apoptosis in the NMDA model and reduced the number of apoptotic cells in tPA/NMDA treated neurons by greater than 80%. Both APC and the caspase-8 inhibitor, z-IETD-fmk, each abolished the tPA-induced increase in caspase-8 activation, while the caspase-9 inhibitor was without effect (FIG. 3B). APC significantly reduced by about 80% (p<0.05) caspase-3 activation in cells exposed to tPA/NMDA (FIG. 3C). A specific caspase-3 inhibitor, Ac-DEVD-CHO, and the caspase-8 inhibitor, but not the caspase-9 inhibitor blocked by greater than 90% caspase-3 activation in tPA/NMDA-treated cells (FIG. 3C). In contrast, the caspase-9 inhibitor greatly reduced (greater than 80%) caspase-3 activation in cells treated with NMDA alone (FIG. 3C). These results suggest that tPA shifts the apoptotic mechanisms during NMDA-mediated neuronal apoptosis to pathways with critical contributions from activated caspase-8.

tPA significantly increased by 50% (p<0.05) nuclear translocation of AIF in the presence of NMDA via a caspase-8 dependent mechanism (FIG. 3D). Namely either caspase-8 or caspase-3 specific inhibitor blocked AIF nuclear translocation in the presence of tPA, while caspase-9 inhibitor was much less effective. Consistent with the inhibition of caspase-8 activation (FIG. 3B), APC blocked AIF mitochondrial to nuclear translocation during NMDA/tPA injury (FIG. 3D). When we assayed tPA's proteolytic effect on the NMDA receptor, tPA failed to cleave either the NR1 or the NR2A subunit of the NMDA receptor (FIG. 3E), in contrast to a previous report⁴ but consistent with a more recent report⁶.

### Example 3

### tPA Neurotoxicity in a Mouse Stroke Model and APC Protection

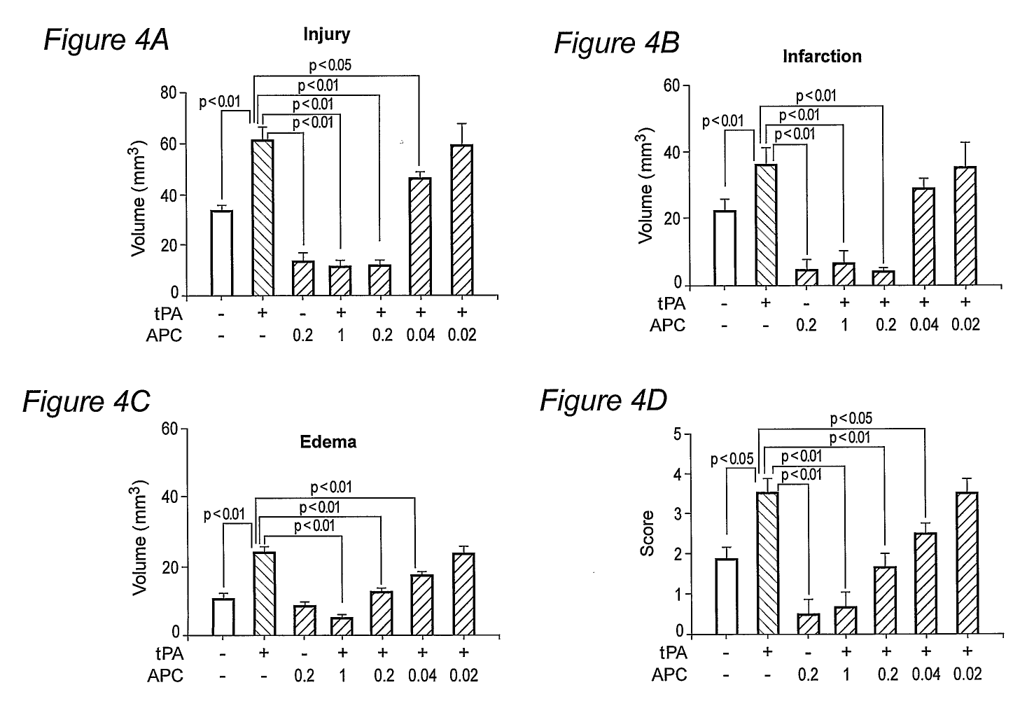

To study whether APC can diminish tPA neurotoxicity in vivo, we have modified our middle cerebral artery (MCA) occlusion model that used a nonsiliconized filament³⁰ and resulted in robust brain damage with secondary brain thrombosis³¹. To cause only moderate brain damage after 24 hr reperfusion, the MCA occlusion was reduced to 45 min (FIGS. 4A-4D). tPA infusion almost doubled the volume of cerebral injury (FIG. 4A), significantly increased the volumes of infarction by 75% (FIG. 4B) and of edema by 155% (FIG. 4C), and correspondingly worsened the motor neurological score from 2.3 to 3.5 (FIG. 4D), compared to controls.

Mouse recombinant APC^{20,32} infused simultaneously with tPA exhibited dose-dependent neuroprotective effects on tPA-induced cerebral injury. At the highest dose studied (1 mg/kg), APC reduced the volumes of total brain injury, infarction, and edema by about 5.0-fold, 6.5-fold, and 3.5-fold, respectively, which was significantly (p<0.05 to 0.01) below the control values in vehicle only-treated animals (FIGS. 4A-4D), and reduced the neurological motor score from 3.5 to 0.6 (FIG. 4D). The lowest dose of APC did not have an effect, while the intermediate dose of 0.04 mg/kg exhibited significant (p<0.05) beneficial effects on brain injury, edema, and neurological score, compared to tPA alone. Infusion of APC (1 mg/kg) after fibrinolytic treatment also resulted in the same remarkable inhibition of undesirable effects of tPA: neuroprotection, reduction in brain injury volume by 4.9-fold, and improvement in the motor neurological score from 3.5 to 0.7. Similar, infusion of APC (1 mg/kg body weight) before fibrinolytic treatment was also neuroprotective and reduced cerebral injury associated with tPA to compare-able levels as simultaneous or post-tPA treatment infusion of APC.

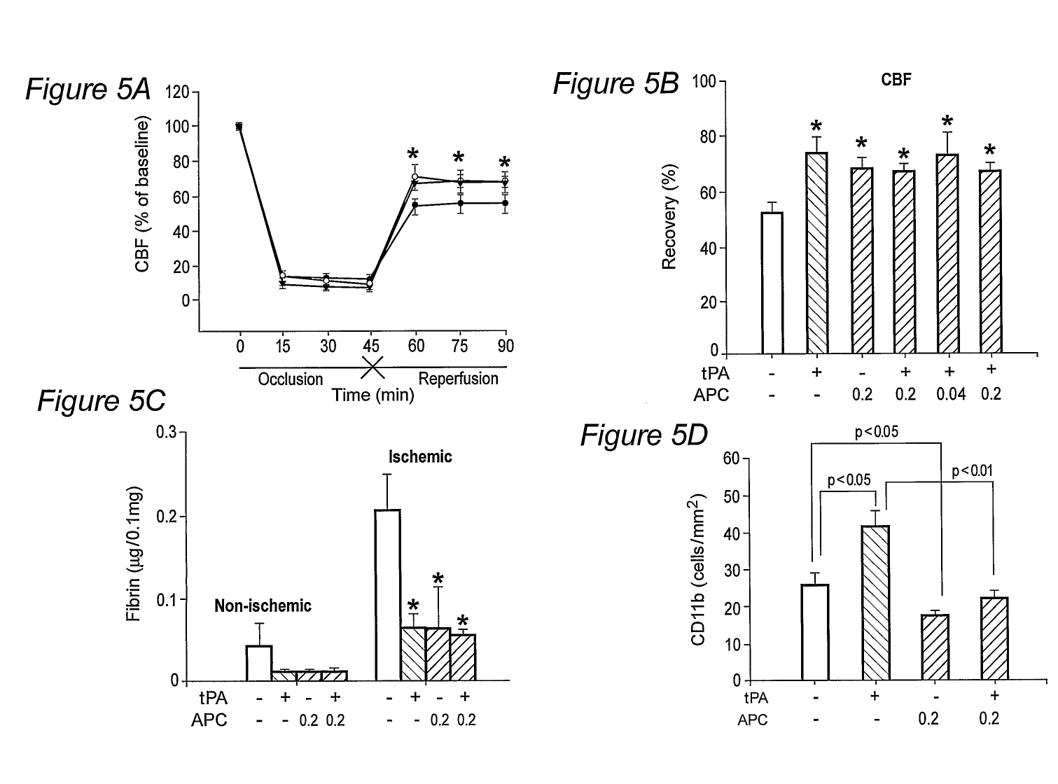

Consistent with its thrombolytic activity, treatment with tPA compared to vehicle only-treated controls significantly improved the post-ischemic cerebral blood flow (CBF) by 35% (FIGS. 5A-5B) and reduced ischemic brain fibrin deposition to the background level (FIG. 5C). Consistent with increased brain injury, however, tPA increased brain accumulation of leukocytes by about 65% (FIG. 5D, confirmed with neutrophil-specific dichloracetate esterase staining).

As expected, APC increased postischemic CBF similar to tPA, but the addition of different doses of APC to tPA did not result in further improvement of CBF which remained at about 70-75% of control baseline values (FIGS. 5A-5B). APC, either alone or with tPA, reduced fibrin levels to the background values (FIG. 5C) and was able to abolish tPA-induced accumulation of neutronphils in ischemic brain (FIG. 5D), which is consistent with its reported blockade of leukocytes transmigration across the BBB mediated by down regulation of intercellular adhesion molecule-1 (ICAM-1)¹⁹.

### Example 4

### tPA-Induced Brain Hemorrhage and MMP-9 Activation Blocked by APC

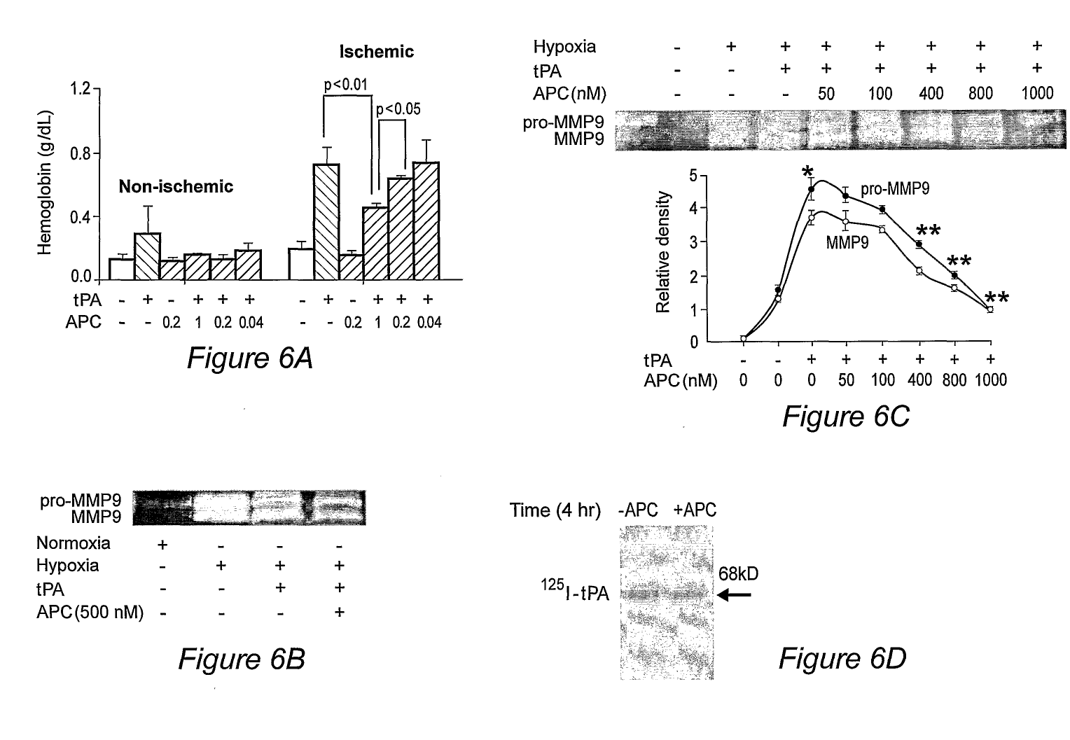

In the MCA stroke model, tPA induced substantial hemorrhage (FIGS. 6A-6B) in all mice studied (6/6). This was reflected in a greater than 3.0-fold increase in the level of hemoglobin in the ischemic hemisphere of animals treated with tPA (FIG. 6A), in contrast to no hemorrhage and barely detectable levels of hemoglobin in ischemic brain tissue in vehicle only-treated controls (FIG. 6A). APC alone (0.2 mg/kg) did not have any effect on intracerebral bleeding or hemoglobin levels in ischemic brain tissue as was previously reported^{15,16,19,20}. At a higher dose, APC (1 mg/kg) reduced the volume of tPA-induced hemorrhage as evidenced by a significant 48% decrease (p<0.01) in the hemoglobin level in an ischemic hemisphere compared to tPA treatment alone, corrected for the residual hemoglobin in brain microvessels (FIG. 6A).

Since activation of MMPs is implicated in the development of tPA-induced hemorrhage¹²⁻¹⁴, we studied the effects of hypoxia and tPA on MMP-9 enzymatic activity in human BEC in the absence or presence of human APC. Hypoxia moderately increased the activation of MMP-9 (FIG. 6B), which was further potentiated by about 2.0-fold by tPA (FIGS. 6B-6C). APC counteracted the tPA-induced increase in MMP-9 activity in hypoxic BEC (FIG. 6B) in a dose-dependent manner (FIG. 6C). That the effect of APC was not due to proteolytic degradation of tPA was confirmed by Western blot analysis demonstrating ¹²⁵I-tPA stability in vitro in the presence of APC (FIG. 6D).

It is generally believed that tPA's intravascular thrombolytic effects are beneficial for stroke therapy, whereas its extravascular effects in the brain, i.e., neurotoxicity and brain hemorrhage, have to be minimized by judicious therapeutic strategies^{9,33}. tPA therapy is limited by a brief 3-hr time window of efficacy³⁴, by its failure sometimes to lyse large clots, reocclusion of arteries in about a third of cases³⁵, and injury to brain cells that may persist despite reperfusion. It has been suggested that tPA exerts direct neurotoxic effects on neurons via cleavage of the NR1 subunit of the NMDA receptor associated with an excessive calcium flux⁴, which potentially might amplify an existing downstream NMDA-mediated apoptotic cascade³⁶. On the other hand, it has also been suggested that tPA-mediated neuronal injury could be secondary to tPA's generation of plasmin⁶, which may promote neuronal death by degrading extracellular matrix proteins such as laminin³⁷⁻³⁹. Nonetheless, plasminogen-independent tPA-induced toxicity was demonstrated in studies with neuroserpin, a natural tPA inhibitor, which can limit tPA-induced neurotoxicity and seizure spreading in vivo⁴⁰. Here, we show that tPA is directly toxic to ischemic brain endothelium and neurons exposed to NMDA, and demonstrate that tPA in these stressed brain cells shifts the driving force for apoptosis from caspase-9 and the intrinsic pathway to a strong requirement for caspase-8 upstream of caspase-3. The caspase-8-dependent apoptotic pathway does not normally play a major role in apoptosis of ischemic BEC, which is dominated by increased p53 and Bax and reduced Bcl-2 expression^{20,41,42} resulting in activation of caspase-9. Caspase-8 is not normally involved in NMDA-mediated cortical neuron apoptosis^{23,26}. The tPA-induced activation of caspase-8 in NMDA-treated neurons results in AIF nuclear translocation that appears to be independent of p53 and caspase-9. Potential mechanisms might include tPA-dependent alterations of “apoptotic threshold” and/or altered crosstalk between the intrinsic and extrinsic pathways.

APC, which inhibits caspase-8 activation in staurosporine-induced path-ways of neuronal apoptosis²³, and the caspase-8 inhibitor z-IETD-fmk, but not the caspase-9 inhibitor z-LEDH-fmk, abolished tPA-induced apoptosis both in ischemic BEC and in NMDA-treated neurons. Consistent with previous reports in BEC^{20,22,43}, the beneficial APC effects required the endothelial protein C receptor (EPCR) and protease activated receptor-1 (PAR1) on BEC and PAR1 and PAR3 on neurons.

In vivo, the beneficial or detrimental effects of tPA critically depend on the type of stroke model³¹ and the time of administration after MCA occlusion⁴⁴. According to earlier reports, tPA reduces neurological damage after cerebral embolism^{45,46}, does not exacerbate ischemic injury⁴⁷, and deletion of the tPA gene in a stroke model with substantial microvascular secondary thrombosis increases the ischemic lesion volume³⁰. In contrast in an MCA model with minimal brain thrombosis³¹, endogenous tPA is directly neurotoxic^{3,5}. Using a modified stroke model in mice with moderate brain injury and secondary thrombosis, we show that tPA worsens both the neurological outcome and neuropathological outcome despite substantial improvement in postischemic reperfusion blood flow and reduced fibrin deposition caused by tPA thrombolytic effect. APC infusion counteracted tPA neurotoxicity in vivo as it did on brain cells in vitro, and APC reduced significantly tPA-induced increases in the infarction and edema volumes, the motor neurological score, and the infiltration of leukocytes.

Although APC is an anticoagulant, APC does not cause bleeding in animal models of sepsis or stroke^{15,16}. Moreover, here we show that APC remarkably attenuates tPA-induced hemorrhage in a mouse stroke model. Recent reports indicate that tPA may open the BBB by acting via low-density lipoprotein receptor-related protein-1 either directly, through intracellular signaling¹⁰ and/or indirectly, by increasing the activity of MMP-9 in BEC and consequent degradation of the vascular basement membrane¹¹. MMP-9 also mediates early disruption of the BBB by reactive oxygen species⁴⁸; and involvement of MMPs in cerebrovascular disease⁴⁹ and tPA-induced hemorrhage including MMP-9 have also been demonstrated¹²⁻¹⁴. Here, we show that hypoxia moderately increases MMP-9 activation and that tPA substantially enhances this activation in hypoxic BEC, whereas APC blocks tPA/hypoxia-induced MMP-9 activation in a dose-dependent manner. These BBB-stabilizing effects of APC required EPCR and PAR1. Thus, it is possible that APC limits tPA-associated hemorrhage by at least two different complementary mechanisms: (i) by preventing tPA-mediated apoptosis of brain endothelial cells which promotes rupture of the BBB in vivo, and (ii) by inhibiting MMP-9 activation which otherwise would proteolytically damage vascular integrity.

In summary, we find that tPA initiates caspase-8-dependent apoptosis in ischemic BEC and in neurons stressed by NMDA. All tPA-induced toxic effects on brain cells both in vitro and in vivo could be blocked by APC in a variety of injury models that include tPA-induced (i) apoptosis in brain endothelium and neurons, (ii) rupture of the BBB, (iii) tPA-induced MMP-9 activity, and (iv) tPA neurotoxicity in a mouse focal ischemia model. Data support our belief that combined tPA/APC therapy, in which tPA reopens occluded blood vessels and dissolves clots, and in which APC inhibits tPA's direct vascular and neuronal toxicities may hold great promise for ischemic stroke therapy.

### REFERENCES

1. NINDS t-PA Stroke Study Group (1995) Tissue plasminogen activator for acute ischemic stroke. New Engl. J. Med. 222:1581-1587.
2. IMS Investigators (2003) The interventional management of stroke (IMS) study: Safety results. Stroke 34:247.
3. Nagai et al. (1999) Role of plasminogen system components in focal cerebral ischemic infarction in gene targeting and gene transfer study in mice. Circulation 99:2440-2444.
4. Nicole et al. (2001) The proteolytic activity of tissue-plasminogen activator enhances NMDA receptor-mediated signaling. Nat. Med. 7:59-64.
5. Wang et al. (1998) Tissue plasminogen activator (tPA) increases neuronal damage after focal cerebral ischemia in wild-type and tPA-deficient mice. Nat. Med. 4:228-231.
6. Matys & Strickland (2003) Tissue plasminogen activator and NMDA receptor cleavage. Nat. Med. 9:371-373.
7. Flavin et al. (2000) Microglial tissue plasminogen activator (tPA) triggers neuronal apoptosis. Glia 29:347-354.
8. NINDS t-PA Stroke Study Group (1997) Intracerebral hemorrhage after intravenous t-PA therapy for ischemic stroke. Stroke 28:2109-2118.
9. Grotta (2003) Adding to the effectiveness of intravenous tissue plasminogen activator for treating acute stroke. Circulation 107:2769-2770.
10. Yepes et al. (2003) Tissue-type plasminogen activator induces opening of the blood-brain barrier via the LDL receptor-related protein. J. Clin. Invest. 112:1533-1540.
11. Wang et al. (2003) Lipoprotein receptor-mediated induction of matrix metalloproteinase by tissue plasminogen activator. Nat. Med 9:1313-1317.
12. Lapchak et al. (2000) Metalloproteinase inhibition reduces thrombolytic (tissue plasminogen activator)-induced hemorrhage after thromboembolic stroke. Stroke 31:3034-3040.
13. Sumii & Lo (2002) Involvement of matrix metalloproteinase in thrombolysis-associated hemorrhagic transformation after embolic focal ischemia in rats. Stroke 33:831-836.
14. Montaner et al. (2003) Matrix metalloproteinase-9 pretreatment level predicts intracranial hemorrhagic complications after thrombolysis in human stroke. Circulation 107:598-603.
15. Griffin et al. (2004) Activated protein C and ischemic stroke. Crit. Care Med. 32:S247-S253.
16. Griffin et al. (2002) Activated protein C: potential therapy for severe sepsis, thrombosis, and stroke. Semin. Hematol. 39:197-205.
17. Bernard et al. (2001) Efficacy and safety of recombinant human activated protein C for severe sepsis. New Engl. J. Med. 344:699-702.
18. Isermann et al. (2003) The thrombomodulin-protein C system is essential for the maintenance of pregnancy. Nat. Med. 9:331-337.
19. Shibata et al. (2001) Anti-inflammatory, antithrombotic and neuroprotective effects of activated protein C in a murine model of focal ischemic stroke. Circulation 103:1799-1805.
20. Cheng et al. (2003) Activated protein C blocks p53-mediated apoptosis in ischemic human brain endothelium and is neuroprotective. Nat. Med. 9:338-342.
21. Joyce et al. (2001) Gene expression profile of antithrombotic protein C defines new mechanisms modulating inflammation and apoptosis. J. Biol. Chem. 276:11199-11203.
22. Riewald et al. (2002) Activation of endothelial cell protease activated receptor 1 by the protein C pathway. Science 296:1880-1882.
23. Guo et al. (2004) Activated protein C prevents neuronal apoptosis via protease activated receptors 1 and 3. Neuron 42:563-572.
24. Kemp & McKernan (2002) NMDA receptor pathways as drug targets. Nature Neurosci. 5:1039-1042.
25. Walford et al. (2004) Hypoxia potentiates nitric oxide-mediated apoptosis in endothelial cells via peroxynitrite-induced activation of mitochondria-dependent and -independent pathways. J. Biol. Chem. 279:4425-4432.
26. Budd et al. (2000) Mitochondrial and extramitochondrial apoptotic signaling pathways in cerebrocortical neurons. Proc. Natl. Acad. Sci., USA 97:6161-6166.
27. Djebaili et al. (2000) p53 and Bax implication in NMDA induced-apoptosis in mouse hippocampus. Neuroreport 11:2973-2976.
28. Uberti et al. (1998) Induction of tumour-suppressor phosphoprotein p53 in the apoptosis of cultured rat cerebellar neurons triggered by excitatory amino acids. Eur. J. Neurosci. 10:246-254.
29. Jordan et al. (2003) Role and regulation of p53 in depolarization-induced neuronal death. Neurosci. 122:707-715.
30. Tabrizi et al. (1999) Tissue plasminogen activator (tPA) deficiency exacerbates cerebrovascular fibrin deposition and brain injury in murine stroke model: studies in tPA-deficient mice and wild-type mice on a matched genetic background. Arterioscler. Thromb. Vasc. Biol. 19:2801-2806.
31. Ginsberg (1999) On ischemic brain injury in genetically altered mice. Arterioscler. Thromb. Vasc. Biol. 19:2581-2583.
32. Fernandez et al. (2003) Recombinant murine activated protein C is neuroprotective in a murine ischemic stroke model. Blood Cell Mol. Dis. 30:271-276.
33. Grotta (2001) Combination therapy stroke trial: recombinant tissue-type plasminogen activator with/without lubeluzole. Cerebrovasc. Dis. 12:258-263.
34. Marler et al. (2000) Early stroke treatment associated with better outcome. Neurology 55:1649-1655.
35. Alexandrov & Grotta (2002) Arterial reocclusion in stroke patients treated with intravenous tissue plasminogen activator. Neurology 59:862-867.
36. Traynelis & Lipton (2001) Is tissue plasminogen activator a threat to neurons? Nat. Med. 7:17-18.
37. Tsirka et al. (1995) Excitotoxin-induced neuronal degeneration and seizure are medicated by tissue plasminogen activator. Nature 377:340-344.
38. Tsirka et al. (1996) Neuronal cell death and tPA. Nature 384:123-124.
39. Chen & Strickland (1997) Neuronal death in the hippocampus is promoted by plasmin-catalyzed degradation of laminin. Cell 91:917-925.
40. Yepes et al. (2002) Regulation of seizure spreading by neuroserpin and tissue-type plasminogen activator is plasminogen-independent. J. Clin. Invest. 109:1571-1578.
41. Stempien-Otero et al. (1999) Mechanisms of hypoxia-induced endothelial cell death. Role of p53 in apoptosis. J. Bio. Chem. 274:8039-8045.
42. Kimura et al. (2001) Orphan G protein-coupled receptor, GPR41, induces apoptosis via a p53/Bax pathway during ischemic hypoxia and reoxygenation. J. Biol. Chem. 276:26453-26460.
43. Mosnier & Griffin (2003) Inhibition of staurosporine-induced apoptosis of endothelial cells by activated protein C requires protease activated receptor-1 and endothelial cell protein C receptor. Biochem. J. 373:65-70.
44. Jiang et al. (2000) Magnetic resonance imaging indexes of therapeutic efficacy of recombinant tissue plasminogen activator treatment of rat at 1 and 4 hours after embolic stroke. J. Cereb. Blood Flow Metab. 20:21-27.
45. Zivin et al. (1985) Tissue plasminogen activator reduces neurological damage after cerebral embolism. Science 230:1289-1292.
46. Overgaurd et al. (1992) Reduction of infarct volume and mortality by thrombolysis in a rat embolic stroke model. Stroke 23:1167-1173.
47. Klein et al. Tissue plasminogen activator does not increase neuronal damage in rat models of global and focal ischemia. Neurology 52:1381-1384.
48. Kim et al. (2003) Neurodegeneration in striatum induced by the mitochondrial toxin 3-nitropropionic acid: role of matrix metalloproteinase-9 in early blood-brain barrier disruption? J. Neurosci. 23:8733-8742.
49. Mun-Bryce & Rosenberg (1998) Matrix metalloproteinases in cerebrovascular disease. J. Cereb. Blood Flow Metab. 18:1163-1172.
50. Zhang et al. (2002) Adjuvant treatment with neuroserpin increases the therapeutic window for tissue-type plasminogen activator administration in a rat model of embolic stroke. Circulation 106:740-745.

Patents, patent applications, books, and other publications cited herein are incorporated by reference in their entirety.

All modifications and substitutions that come within the meaning of the claims and the range of their legal equivalents are to be embraced within their scope. From the foregoing, it would be apparent to a person of skill in this art that the invention can be embodied in other specific forms without departing from its spirit or essential characteristics. For example, variants of activated protein C are known as homologs, mutations, and polymorphisms in the known nucleotide and amino acid sequences.

A claim using the transition “comprising” allows the inclusion of other elements to be within the scope of the claim; the invention is also described by such claims using the transitional phrase “consisting essentially of” (i.e., allowing the inclusion of other elements to be within the scope of the claim if they do not materially affect operation of the invention) and the transition “consisting” (i.e., allowing only the elements listed in the claim other than impurities or inconsequential activities which are ordinarily associated with the invention) instead of the “comprising” term. Any of these three transitions can be used to claim the invention.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements disclosed herein are considered to be aspects of the invention. Similarly, generalizations of the invention's description are considered to be part of the invention.

It should be understood that an element described in this specification should not be construed as a limitation of the claimed invention unless it is explicitly recited in the claims. Thus, the described embodiments should be considered only as illustrative, not restrictive, because the scope of the legal protection provided for the invention will be indicated by the appended claims rather than by this specification.

1. A method of inhibiting deleterious effects of plasminogen activator in a subject's brain, said method comprising:
(a) treating a subject with a plasminogen activator, and
(b) administering a pharmaceutical composition comprising an effective amount of activated protein C, at least one prodrug, or at least one functional variant thereof to said subject such deleterious effects of plasminogen activator is inhibited.

2. The method of claim 1, wherein said plasminogen activator is administered to said subject to treat stroke.

3. The method of claim 2, wherein said plasminogen activator is administered to said subject within 3 hours of said stroke.

4. The method of claim 1, wherein said plasminogen activator is administered to said subject in an amount of at least 0.9 milligrams per kilogram body weight of said subject.

5. The method of claim 1, wherein said pharmaceutical composition is administered to said subject within 24 hours of said treatment with plasminogen activator.

6. The method of claim 1, wherein said effective amount of activated protein C is from 0.005 milligrams to 2 milligrams per kilogram body weight of said subject, or an equivalent amount of said prodrug or said functional variant.

7. The method of claim 1, wherein at least brain hemorrhage, conversion of ischemic stroke to hemorrhagic stroke, tissue damage, or a combination thereof is reduced.

8. The method of claim 1, wherein at least apoptosis or cell death in the brain is reduced.

9. The method of claim 1, wherein said pharmaceutical composition is adapted for delivery to the brain.

10. The method of claim 1, wherein said pharmaceutical composition is administered before treatment with plasminogen activator.

11. The method of claim 10, wherein said pharmaceutical composition is administered at least 30 minutes before treatment with plasminogen activator.

12. The method of claim 1, wherein said pharmaceutical composition is administered after treatment with plasminogen activator.

13. The method of claim 12, wherein said pharmaceutical composition is administered at least 30 minutes after treatment with plasminogen activator.

14. The method of claim 1, wherein said pharmaceutical composition is administered at the approximately the same time as treatment with plasminogen activator.

15. The method of claim 1, wherein said activated protein C, at least one prodrug, or at least one variant thereof is derived from human.

16. The method of claim 1, wherein said subject is human.

17. (canceled)

18. A process of screening for an agent, said process comprising:
(a) providing a library of candidate agents which are variants of activated protein C and/or protein C,
(b) treating a subject or cells with a plasminogen activator, and
(c) selecting at least one agent from said library by its ability to reduce deleterious effects due to said plasminogen activator.

19. A process of screening for an agent, said process comprising:
(a) providing a library of candidate agents which are variants of activated protein C and/or protein C,
(b) treating a subject or cells with a plasminogen activator, and
(c) selecting at least one agent from said library by its ability to inhibit caspase-8 signaling and/or expression of matrix metalloproteinase-9 in said subject or cells.

20. An agent produced by the process of claim 18.
